(19) United States
(12) Patent Application Publication
(10) Pub. No.: US 2008/0050456 A1
(43) Pub. Date: Feb. 28, 2008

(54) Intramammary formulations

(76) Inventor: Wayne Frederick Leech, Auckland (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 11/508,357

(22) Filed: Aug. 23, 2006

(51) Int. Cl. A61K 36/886; A61K 36/28; A61K 31/43

(52) U.S. Cl. 424/744; 424/764; 514/192

(57) ABSTRACT

The present invention relates to intramammary formulations, as well as methods of treating an intra-mammary infection in a lactating non-human animal and preventing re-infection by internal administration of the formulations. The formulations include a combination of penicillin, aloe vera and Centella asiatica extract.

### FIELD OF THE INVENTION

The present invention relates to intramammary formulations, as well as methods and uses of the formulations. More specifically, the invention relates to formulations and associated methods of treatment and uses of the formulations for intramammary administration containing a combination of at least one antibiotic and at least one natural product or extract.

### DESCRIPTION OF THE RELATED ART

Intramammary formulations have been considered for use in the treatment of various afflictions associated with the mammary gland. One major use for such formulations is in the treatment of lactating animals such as cows.

Existing methods of application include infusions that are inserted into the mammary gland via the teat; injection formulations; and topically administered formulations such as creams, lotions and ointments.

One affliction associated with intramammary infection in lactating animals, particularly lactating cows, is mastitis.

For the purposes of this discussion, further illustration will be made with respect to mastitis however, this should not be seen as limiting as other mammary gland related afflictions may also be encompassed within the present invention.

Mastitis is classed as sub-clinical or clinical in nature depending on the degree of infection. It is largely caused by microbial infection of the mammary gland. Milk obtained from cows affected by clinical mastitis has an acidic off taste and is not able to be used for milk production. Sub-clinical mastitis is usually first noticed by lowered levels of milk production. Both types of mastitis can represent significant costs to the dairy farmer and industry as a whole as it results in lost or reduced milk production. Further, if mastitis tainted milk is inadvertently mixed into full production runs, whole production runs may need to be discarded.

A further problem is that dairy cows that have had mastitis are often more sensitive to re-infection, either in that same season, or earlier in the following season. It is understood by the inventors that this higher sensitivity is because the tissue at the original infection site is prone to re-infection due to permanent damage. It is suspected that heifers (first lactation heifers) developing mastitis in their first lactation can suffer lasting damage to the udder tissue and a lifetime reduction in milk production due to mastitis re-infection.

One main method of treating mastitis infection is to administer antibiotics to the mammary gland. However, the efficacy of antibiotic treatments may decrease over time as re-infection occurs more frequently due to residual tissue damage. Further problems associated with antibiotic treatment include:

The negative public perception against use of antibiotics;

The increasing resistance of microbes to antibiotics;

The cost of antibiotic treatments; and,

The antibiotic treatment only addresses the current infection and not aggravating or underlying factors of mastitis such as tissue damage.

Given the problems associated with antibiotic treatments, alternative therapies using natural products have been considered.

For example, one reference (Singh, S. V. et al. “Antibiotic therapy in bovine mastitis: a double-edged sword” Pashudhan Jnl vol. 16, No. 8 (2001) pp. 1) suggests that the use of micronutrients including selenium, zinc, copper, vitamins A, E and C, herbal drugs and cytokines, aid in the control and prevention of mastitis.

Prior art existing treatments known to the inventors that incorporate natural products or extracts are largely split between treatment of sub-clinical and clinical mastitis.

Three references relating to sub-clinical mastitis treatments (Bogdan, L. M. “A new diagnostic method and treatment of sub-clinical mastitis in dairy cows” Lucrai Stiinifice-Medicina Veterinara, Universitatea de Stiinte Agricole si Medicina Veterinara “Ion Ionescu de la Brad” lasi, Vol. 44 No. 3(2) (2001) pp. 477-480; Kolte, A. Y. et al. “Comparative efficacy of indigenous medicinal plant preparation and tilox in sub-clinical mastitis in cows” Indian Veterinary Journal, Vol. 76, No. 10, (1999) pp. 893-895; Abaineh, D. et al, “Treatment trial of sub-clinical mastitis with the herb Persicaria senegalense (Polygonaceae)” Trap Anim Health Prod. Vol. 33, (2001) Dec pp. 511-519) relate to comparison studies between antibiotic treatments compared with three different forms of natural product treatment. In general, therapeutic effects were found for both antibiotic treatment and the natural products tested however, the natural remedies tended to take longer to be effective or require additional applications.

One reference (Rahman H. et al. “Efficacy of Mastilep as supportive therapy for clinical mastitis in cows” Indian Veterinary Journal, vol. 77, No. 1, (2000), pp. 50-52) relating to clinical mastitis treatment describes a combination therapy using a herbal gel with antibiotic treatment. The study found that the combination provided an improved efficacy compared with using either antibiotics or the gel alone.

Two other clinical mastitis studies (Liu H. et al. “Clinical study of Chrysanthemum indicum preparations in the treatment of bovine mastitis” Journal of Zhejiang Agricultural University, Vol. 22, No. 6, (1996), pp. 643-646; Fang W. H. et al. “The non-antibiotic approach to the treatment of bovine mastitis” Proceedings of World Association of Veterinary Food Hygienists Xth (Jubilee) International Symposium in Stockholm, 2-7 Jul. 1989 pp. 281-284) compare the use of two further types of natural product against antibiotic treatment. In both cases, the natural product was found to have a similar or slightly reduced therapeutic effect than antibiotic treatment.

One further reference (Du A. et al. “Study of houttuyin sodium bisulphate for bovine clinical mastitis therapy” Acta Agriculturae Zhejiangensis, Vol. 9, No. 3, (1997), pp. 165-168) describes an intramammary infusion of houttuynin sodium bisulphate (obtained from Houttuynia cordata) compared to treatment with antibiotics. A roughly equivalent cure rate is described as having been achieved for both methods in treating acute (clinical) and sub-acute (sub-clinical) mastitis.

Problems with the above treatments are that:
The natural ingredients used may be unusual and therefore difficult to obtain in commercial quantities;
Have a limited raw material supply e.g., through small growing habitats;
Require specific or complicated administration regimes not practical in commercial applications such as on farms;
Require the use of costly and/or complex extraction or formulation processes to obtain the natural ingredients used for the treatment formulation.

Two natural products used in related fields such as for wound healing, to reduce inflammation and to fight infection, include Centella asiatica and aloe vera.

Centella asiatica is a saponin type plant from the Umbelliferae family that is widely used in medicinal treatments for a variety of applications due to its wound healing and anti-inflammatory properties (World-wide web at: ayuherbal.com/herboftheweek.htm (30 Apr. 2004)). Other uses include in-vivo medicaments for liver cell treatment (U.S. Pat. No. 6,417,349) and cosmetic formulations (U.S. Pat. No. 6,361,804 and U.S. Pat. No. 6,261,605). Wound healing medicaments including Centella asiatica have been considered in at least U.S. Pat. No. 6,267,996 and asiatica extracts such as asiatic acid in at least U.S. Pat. No. 5,834,437. In U.S. Pat. No. 6,267,996, one embodiment uses a formulation comprising a wide variety of natural plant extracts including Centella asiatica as an acne treatment. Whilst Centella asiatica appears as only a minor component, it is however purported as having some effect as an anti-acne and hence an anti-bacterial agent.

Aloe vera is a member of the lily family although it more closely resembles cactus plants in its characteristics. Of the 240+ species of aloe, four are recognised as having nutritional value with Aloe barbadensis leading this group. Aloe barbadensis is the type of aloe used in most aloe containing products available commercially (World-wide web at: aloe-vera.org).

The aloe leaf also contains over 75 nutrients and 200 active compounds including 20 minerals, 18 amino acids and 12 vitamins. Aloe vera is used in a wide variety of applications including moisturisers, herbal beverages, dietary supplements and is used in cosmetic products more than any other herb (World-wide web at: iasc.org/aloerx.html).

In veterinary medicine, aloe vera is used as an approved drug to treat feline leukaemia and tumours in dogs and cats (World-wide web at: iasc.org/aloerx.html). Other studies have identified that:

Aloe may reduce inflammation, decrease swelling and redness and wound healing (vulnerary) (World-wide web at: kcweb.com/herb/aloevera.htm);

Aloe is an emollient with soothing, softening properties (World-wide web at: egregore.com/herbs/aloevera.html);
Aloe has demulcent properties i.e., soothing and protective (World-wide web at: egregore.com/herbs/aloevera.html);
Aloe has alterative properties i.e., tending to restore to normal health (World-wide web at: egregore.com/herbs/aloevera.html);
Consuming aloe vera with vitamin E or C increased the half-life of the vitamin in the plasma (World-wide web at: iasc.org/aloerx.html).

The above natural products show promise in terms of selected wound healing applications, particularly for topical preparations. Less consideration appears to have been given to other methods of administration including intra-mammary treatments.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e., that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY OF THE INVENTION

One embodiment of the invention relates to a method of treating an intra-mammary infection in a lactating non-human animal and preventing re-infection, by internal administration of a composition comprising penicillin, aloe vera and Centella asiatica to the mammary gland of the non-human animal.

Another embodiment of the invention relates to a method of preventing microbial infection and healing internal wounds in the mammary gland of a non-human animal by administration of a composition comprising penicillin, aloe vera and Centella asiatica inside the mammary gland of the non-human animal.

Another embodiment of the invention relates to a medicament comprising penicillin, aloe vera and Centella asiatica.

### DETAILED DESCRIPTION OF THE PREFERRED EMBODIMENT

For the purposes of this specification, the term ‘extract’ is used loosely to refer to the fact that the botanical component of the composition is processed in a manner beyond its natural physical form. More preferably, the term ‘extract’ refers to a powdered form of the botanical product. However, it should be appreciated that the term ‘extract’ may also encompass liquid forms of the botanical product or a specific portion or portions thereof.

For the purposes of this specification, the term ‘antibiotic’ refers to any chemical substance which has the capacity to inhibit the growth of, or to kill, other microorganisms.

The term ‘botanical’ refers to the product or extract being plant based.

The term ‘intramammary’ refers to inside or within the mammary gland of an animal including the teat canal.

The terms ‘mammary’, ‘mammary gland’ and ‘udder’ refer to the milk producing gland of female mammals.

According to one aspect of the present invention there is provided a method of promoting mammary gland health in an animal by administration of a composition including a combination of:

at least one antibiotic with;

an aloe vera extract; and,

a Centella asiatica extract;

formulated for intra-mammary administration.

According to a further aspect of the present invention there is provided a method of preventing mammary microbial infection in an animal by administration of a composition substantially as described above.

According to a further aspect of the present invention there is provided a method of treatment of mastitis in an animal by administration of a composition substantially as described above.

According to a further aspect of the present invention there is provided a method of treating damaged tissue in the mammary gland of an animal by administration of a composition substantially as described above.

According to a further aspect of the present invention there is provided the use of at least one antibiotic; an aloe vera extract and a Centella asiatica extract in the manufacture of a medicament for the promotion of mammary gland health in an animal

According to a further aspect of the present invention there is provided the use of a composition substantially as described above in the manufacture of a medicament for preventing mammary gland microbial infection in an animal.

According to a further aspect of the present invention there is provided the use of a composition substantially as described above in the manufacture of a medicament for treatment of mastitis in an animal.

According to a further aspect of the present invention there is provided the use of a composition substantially as described above in the manufacture of a medicament for treating damaged tissue in the mammary gland of an animal.

In preferred embodiments the animal maybe a lactating cow. However, this should not be seen as limiting as it should be appreciated by those skilled in the art that the composition of the present invention could also be administered to other lactating animals (including humans).

It has been found by the inventors that the combination of at least one antibiotic and at least one natural product and/or extract such as aloe vera and Centella asiatica provides an unexpected and even synergistic effect on treatment and prevention of mammary gland related health problems including, but not limited to, treatment and prevention of microbial infection.

It is understood by the inventors that the composition of the present invention by promotes udder health generally and addresses more than just a single microbial infection event as in standard antibiotic treatments. This is thought to be because pathogenic microbes are always present within the mammary gland and, even when treated with antibiotics, active microbes, antibiotic resistant microbes and/or microbe spores, may still be present. These microbes re-infect the gland, particularly on or adjacent damaged tissue sites left from an earlier infection or other event. Therefore, by treating both pathogenic microbes and addressing other aspects of udder health including tissue regeneration and reducing scarring, the composition of the present invention provides not only an infection treatment remedy, but also an infection prevention remedy. It is also envisaged by the inventors that the composition reduces pain and inflammation associated with microbial infection and tissue damage of the mammary gland.

In preferred embodiments, the composition, substantially as described above, maybe administered as a mastitis treatment.

More preferably, the mastitis to be treated maybe either sub-clinical mastitis or clinical mastitis. It should be appreciated that dosages and dosage regimes may be altered depending on the level of mastitis to be treated.

It is understood by the inventors that dose rates may also vary depending on the metabolism level, age, weight, species or genetics of the animal and other biochemical factors, such as seasonal dietary requirements.

Preferably the natural compound may have an anti-inflammatory effect.

Preferably the natural compounds may be products or extracts selected from: Centella asiatica and aloe vera although may also include arnica; ginger; salicylates; rosemary; skullcap; licorice; and combinations thereof.

More preferably, the natural compounds may be extracts from Centella asiatica and aloe vera.

Preferably, where Centella asiatica is used, the extract may be from either the leaf of the plant or the whole plant.

Preferably, where aloe vera is used, the centre fillet of Aloe barbadensis may be used which may be processed by cold pressing to give a single strength gel concentrated through evaporation to a ratio of 200:1 and then freeze dried.

Preferably, the antibiotics used may be cephalosporins. More preferably, the antibiotics used are penicillin related antibiotics. Most preferably, the antibiotic may be penicillin.

In preferred embodiments and to maintain a natural product image, the antibiotic may be naturally derived and not produced synthetically. This should not be seen as limiting as it should be appreciated by those skilled in the art that synthetic antibiotics may also be used without departing from the scope of the invention as described above.

In one preferred embodiment, the composition used may be an infusion that includes: penicillin, aloe vera extract and Centella asiatica extract. More preferably, the composition contains approximately 1 gram of penicillin, 0.05% wt of aloe vera extract and 0.5% wt Centella asiatica extract. Although quantities have been described, these should not be seen as limiting as it should be appreciated by those skilled in the art that quantities outside of these levels may also be used without departing from the scope of the invention as described above.

It should also be appreciated by those skilled in the art that dosages of at least the natural components of the composition may be greater than that described above without causing harm to the animal. Most natural products and extracts are generally non-toxic. Higher doses are unlikely to produce any toxic reactions to the animal and may in fact be advantageous for some animals that require nutritional treatment.

In a further embodiment, the composition, substantially as described above, may also be formulated using components selected from: fillers; excipients; modifiers; humectants; stabilisers; emulsifiers; and other known formulation components.

In preferred embodiments the composition may be administered via one single dose method e.g., as an infusion or as a combination administration, for example, the natural product or extract may be applied topically and the antibiotic may be inserted into the teat as an infusion. For the purposes of the remainder of this specification, administration will be described in terms of a single dose rather than separate methods of administration. This should not be seen as limiting as it should be appreciated by those skilled the art that other combinations of dose may also be possible without departing for the scope of the invention.

Preferably, the composition, substantially as described above, may be administered in a form selected from: an infusion; a tablet; a capsule; a suppository; an injection; a suspension; a drink or tonic; a syrup; a powder; an ingredient in solid or liquid foods; a nasal spray; a sublingual wafer; a transdermal patch; a transdermal injection; a topical solution; and combinations thereof.

Preferably, the composition may be administered as an infusion applied via the mammary gland teat or topically applied to the surface of the mammary gland or an animal.

Preferably, in the infusion embodiment, the composition may be administered into the animal teat via an applicator or similar device which may be inserted into the teat opening. In a preferred embodiment, it is envisaged that the infusion may be squeezed from the applicator by hand (e.g., by squeezing the infusion out of a deformable tube) and subsequently massaging the infusion by hand up the teat and into the mammary gland.

Preferably, where the composition is administered topically, the composition may be formulated as a cream. It should be appreciated that alternative topical formulations are also possible including ointments and lotions without departing from the scope of the invention. Cream formulations are preferred as they are relatively simple to formulate and administer, particularly to an animal body surface such as skin rather than having a poultice which must be held in place, or a powder which is easily rubbed off by the animal.

Preferably, the topical cream may be applied, typically via rubbing, onto the surface skin of the mammary gland and/or teat of an animal.

In preferred embodiments, pharmaceutically and physiologically acceptable ingredients may be added to the active ingredients to obtain a desired formulation.

In preferred embodiments, pharmaceutically and physiologically acceptable ingredients are selected from at least one: oil; wax; acid; viscosity control agent; pigment dispersing agent; emulsifier; stabiliser; humectant; antioxidant; water; and combinations thereof.

It should be appreciated from the above description that there is provided a composition that may provide: improved effectiveness against microbial infection; improved healing of tissue damage including minimising, preventing or treating internal scarring.

An advantage of the present invention may be a decrease in the likelihood of reinfection occurring due to a reduction in scar sites.

A further advantage for dairy production is that as the composition treats mastitis faster than antibiotic treatments alone:
Future re-infection is prevented or reduced;
Less time is required to withhold milk production due to use of natural products; and,
Cows are out of production less often, higher yields of milk production result.

A further advantage of the present invention is that the composition is largely natural and as a result it may be more readily adopted and absorbed by the body with fewer side effects. Also the dependency on antibiotics may be reduced as the composition may have a preventative effect and will therefore reduce the likelihood of further antibiotics needing to be administered.

The composition therefore overcomes disadvantages associated with the use of antibiotics and the need for specialised natural products or extraction processes, whilst at the same time providing a homogenous, easy to use preparation for intramammary treatments.

### EXAMPLE

The invention will now be described with reference to examples of preferred formulations known to the inventors.

For the purposes of the examples below, reference will be made to use of penicillin, and extracts from aloe vera and Centella asiatica. This should not be seen as limiting as it should be appreciated by those skilled in the art that other types of antibiotic and/or natural ingredients may also be used in accordance with the present invention.

The formulation is made up of:
Penicillin antibiotic (1 g),
Aloe vera extract (0.05% wt) prepared by cold pressing to give a single strength gel concentrated through evaporation to a ratio of 200:1 and then freeze dried;
Centella asiatica extract 0.5% wt.

The formulation of a single infusion is administered via a tube applicator into the teat of a lactating cow. The applicator is inserted approximately 1 cm into the teat and the contents of the infusion extracted from the applicator e.g., by squeezing a tube so that the infusion enters the teat. Then, by hand, the infusion is massaged up the teat and into the mammary gland.

It is envisaged that the infusion is reapplied on a preventative regular basis or when infection is detected.

Aspects of the present invention have been described by way of example only and it should be appreciated that modifications and additions may be made thereto without departing from the scope thereof as defined in the appended claims.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method of treating an intra-mammary infection in a lactating non-human animal and preventing re-infection, by internal administration of the medicament according to claim 8 to the mammary gland of the non-human animal.

2. A method of preventing microbial infection and healing internal wounds in the mammary gland of a non-human animal by administration of the medicament according to claim 8 inside the mammary gland of the non-human animal.

3. The method as claimed in claim 1 or claim 2 wherein the infection is associated with mastitis.

4. The method as claimed in claim 1 or claim 2 wherein the nonhuman animal is a lactating cow.

5. The method as claimed in claim 1 or claim 2 wherein the composition is formulated as an infusion administered via the mammary gland teat.

6. The method as claimed in claim 5 wherein the infusion comprises approximately 1 gram of penicillin, 0.05% wt of aloe vera extract and 0.5% wt Centella asiatica extract.

7. The method as claimed in claim 1 or claim 2 wherein the composition further comprises pharmaceutically and physiologically acceptable ingredients selected from the group consisting of: oil; wax; acid; viscosity control agent; pigment dispersing agent; emulsifier; stabiliser; humectant; antioxidant; water; and combinations thereof.

8. A medicament comprising penicillin, aloe vera and Centella asiatica extract.

9. The medicament as claimed in claim 8 wherein the composition is formulated as an infusion for administration via the mammary gland teat

10. The medicament as claimed in claim 9 wherein the infusion comprises approximately 1 gram of penicillin, 0.05% wt of aloe vera extract and 0.5% wt Centella asiatica extract

11. The medicament as claimed in claim 8 wherein the composition further comprises pharmaceutically and physiologically acceptable ingredients selected from the group consisting of: oil; wax; acid; viscosity control agent; pigment dispersing agent; emulsifier; stabiliser; humectant; antioxidant; water; and combinations thereof.
